(19) United States
(12) Patent Application Publication
(10) Pub. No.: US 2022/0005580 A1
(43) Pub. Date: Jan. 6, 2022

(54) METHOD FOR PROVIDING RECOMMENDATIONS FOR MAINTAINING A HEALTHY LIFESTYLE BASING ON DAILY ACTIVITY PARAMETERS OF USER, AUTOMATICALLY TRACKED IN REAL TIME, AND CORRESPONDING SYSTEM

(71) Applicant: Samsung Electronics Co., Ltd., Suwon-si, Gyeonggi-do (KR)

(72) Inventors: Konstantin Aleksandrovich PAVLOV, Moscow (RU); Alexey Viacheslavovich PERCHIK, Moscow (RU); Vladislav Valerievich LYCHAGOV, Saratov (RU); Hyejung SEO, Suwon-si (KR); Minji KIM, Suwon-si (KR)

(21) Appl. No.: 17/297,189

(22) PCT Filed: Nov. 27, 2019
(86) PCT No.: PCT/KR2019/016501

§ 371 Date: May 26, 2021

(30) Foreign Application Priority Data
Nov. 29, 2018 (RU) 2018142204
Nov. 27, 2019 (KR) 10-2019-0154335

(51) Int. Cl. G16H 20/70; A61B 5/00; A61B 5/0205; G16H 20/60; G16H 50/30; G16H 40/67

(52) U.S. Cl. CPC G16H 20/70; A61B 5/7267; A61B 5/0205; A61B 5/742; A61B 5/14532; G16H 20/60; G16H 50/30; G16H 40/67; A61B 5/486

(57) ABSTRACT

According to a first aspect of the present invention, there is provided a method for providing recommendations for maintaining a healthy lifestyle basing on user's daily activity parameters automatically tracked in real time, comprising the steps of: measuring automatically the user's daily activity parameters, including periods of physical activity, changes in blood glucose level, and data of a food intake; building a physiological model basing on the measured change in the user's blood glucose level to determine an individual response of the user to food intake; training a machine learning algorithm to estimate the user's daily activity basing on the measured parameters of the user's daily activity, the determined individual response of the user and a predefined user profile containing the user's gender, age, height and weight; generating recommendations for maintaining of the user's healthy lifestyle basing on estimation of the unser's daily activity received as a result of using the machine learning algorithm; and displaying generated recommendations to the user.

### TECHNICAL FIELD

The present group of inventions relates to the field of tracking a user's daily activity and, in particular, to a method and system for providing recommendations for maintaining a healthy lifestyle basing on user's daily activity parameters, automatically tracked in real time.

### BACKGROUND ART

Currently, there are a huge number of solutions that contribute to maintaining the user's health and physical form. As a rule, these known solutions are based on the analysis of various aspects of the user's daily life and/or vital signs of a human body.

In particular, a prior art solution is known, disclosed in US 20150364057 A1 (“Systems and methods for wellness, health, and lifestyle planning, tracking, and maintenance”), which describes systems and methods for healthy lifestyle planning, tracking, and maintenance. The known system allows a person to manage his lifestyle and healthy habits. In an exemplary embodiment, this system can be configured to provide recommendations of activities to the user that can positively affect the user's a wellness, health, and lifestyle. The recommendations can be tailored to each individual user of the system such that different people can receive different recommendations. However, this system does not contain any means for automatically tracking the parameters of the user's health or wellness. In addition, for it to work, the user must input data for analysis manually, which entails not only the possibility of inputting erroneous data, but also the likelihood that the user will forget to input data, or he will be tired of doing it.

There is also known a prior art solution, disclosed in U.S. Pat. No. 8,182,424 B2 (“Diary-free calorimeter”), which discloses an indirect calorimeter (i.e. with indirect instrumental measurement), which estimates nutritional caloric intake by periodically monitoring user's weight and sensing physical exercise (i.e., physiological data and/or motion data related to physical exertion). A user device according to this solution can detect one or more of heart rate, body temperature, skin resistance, motion/acceleration sensing (e.g., pedometer, accelerometer), velocity sensing (e.g., global positioning system (GPS)).

However, this system does not provide recommendations regarding physical activity or sleep efficiency. Moreover, its correct functioning requires, as indicated above, mass measurements that are not automatically performed by the system (but must be inputted by the user), as well as tracking long-term changes.

A solution U.S. Pat. No. 9,569,483 B2 (“Personalized dynamic feedback control of body weight”) discloses a personalized weight management system incorporating feedback control, using a mathematical model of metabolism and weight change. In particular, this system provides monitoring of such parameters as, for example, body weight, physical activity, diet, eating behavior etc. However, this known solution also does not imply any means for automatic monitoring of these parameters, but requires manual input of the necessary information by the user, which entails not only the possibility of inputting erroneous data, but also the likelihood that the user will forget to input data, or he will be tired of doing it.

A prior art solution is also known, disclosed in U.S. Pat. No. 8,706,731 B2 (“System and method for providing healthcare program service based on vital signals and condition information”), which describes a method for providing a healthcare program service over a wireless communication network, which includes: receiving vital signals for condition transmitted from multiple users, grouping the received vital signals for condition, registering the corresponding healthcare programs classified by particular diseases, and providing a healthcare program service to the users. Meanwhile, this solution does not disclose any specific methods for processing data, grouping the users and selecting an appropriate program to maintain a healthy lifestyle. In addition, this solution does not involve the use of physiological models to improve the recommendations provided by programs and also requires manual input of some necessary data by the user.

An artificial intelligence system is also known (see US 20180108272 A1, “Artificial intelligence based health coaching based on ketone levels of participants”), that uses profiles of users, including monitored ketone levels of the users, to assess effectiveness levels of health programs (such as weight loss programs). However, this system includes a breath analysis device in which the user needs to breathe in order to determine the user's ketone level, which is not an automatic process, but a user-dependent one. In addition, this solution does not imply the use of physiological models to improve provided program recommendations as well.

A solution disclosed in document US 20160262693 A1 (“Metabolic analyzer for optimizing health and weight management”), describes a system including a metabolic rate monitor that can monitor one or more metabolic determinants to determine a user's metabolic rate. An interval identifier can detect a plurality of intervals corresponding to a least one type of user activity over a time period. However, no sources of required data (i.e., means of receiving the data) are defined in this document. In addition, this solution also does not imply the use of physiological models to improve provided program recommendations.

The closest prior art of the claimed group of inventions is the solution disclosed in U.S. Pat. No. 9,675,289 B2 (“Method and glucose monitoring system for monitoring individual metabolic response and for generating nutritional feedback”). This solution describes a system and a method for monitoring individual metabolic response and for generating nutritional feedback, that comprise monitoring of a glucose level in a subject. However, this solution does not provide recommendations regarding physical activity and sleep efficiency, which are also important criteria for a healthy lifestyle. In addition, this solution does not imply the use of physiological models to improve the recommendations provided by programs and analysis of data collected from multiple individuals.

Thus, there is a need for a fully automatic method for tracking user's daily activity and providing appropriate recommendations to the user, which comprises using physiological models to improve recommendations provided by the programs.

### DISCLOSURE OF INVENTION

### Technical Problem

The object of the present invention is to eliminate the above-mentioned disadvantages inherent in prior art solutions, in particular, to provide an improved method for tracking user's daily activity in a user-independent mode and providing appropriate recommendations to the user on maintaining a healthy lifestyle.

This object is solved by means of methods and systems that are characterized in the independent claims. Additional embodiments of the present invention are presented in the dependent claims.

### Solution to Problem

The object of the present invention is to eliminate the above-mentioned disadvantages inherent in prior art solutions, in particular, to provide an improved method for tracking user's daily activity in a user-independent mode and providing appropriate recommendations to the user on maintaining a healthy lifestyle.

This object is solved by means of methods and systems that are characterized in the independent claims. Additional embodiments of the present invention are presented in the dependent claims.

According to a first aspect of the present invention, there is provided a method for providing recommendations for maintaining a healthy lifestyle basing on user's daily activity parameters automatically tracked in real time, comprising the steps of:
measuring automatically the user's daily activity parameters, including periods of physical activity, heart rate, the number of steps taken, a sleep time period, changes in blood glucose level, the amount of carbohydrates and calories taken with food;
building a physiological model basing on the measured change in the user's blood glucose level to determine an individual response of the user to food intake;
training a machine learning algorithm to estimate the user's daily activity basing on the measured parameters of the user's daily activity, the determined individual response of the user and a predefined user profile containing the user's gender, age, height and weight;
generating recommendations for maintaining of the user's healthy lifestyle basing on estimation of the user's daily activity received as a result of using the machine learning algorithm; and
displaying generated recommendations to the user.

According to another aspect of the present invention, there is provided a system for providing recommendations for maintaining a healthy lifestyle basing on user's daily activity parameters automatically tracked in real time, comprising:
inertial measuring sensors, including an accelerometer and a gyroscope;
a photoplethysmogram sensor;
a blood glucose sensor,

wherein the inertial measuring sensors, the photoplethysmogram sensor and the blood glucose sensor are configured to automatically measure the user's daily activity parameters, including periods of physical activity, heart rate, the number of steps taken, a sleep time period, changes in blood glucose level, the amount of carbohydrates and calories taken with food;
a processing unit configured to build a physiological model basing on a change in the user's blood glucose level to determine an individual response of the user to food intake and training a machine learning algorithm to estimate the user's daily activity basing on the measured parameters of the user's daily activity, the determined individual response of the user, and a predefined user profile containing the user's gender, age, height and weight;
a storage module configured to store the predefined user profile, the measured parameters of the user's daily activity, the determined individual response of the user and estimation of the user's daily activity received as a result of using the machine learning algorithm,

wherein the processing unit is additionally configured to generate recommendations for maintaining of the user's a healthy lifestyle basing on estimation of the user's daily activity, and the storage module is configured to store the generated recommendations,

wherein the system for providing recommendations for maintaining a healthy lifestyle basing on the user's daily activity parameters further comprises a display configured to display the generated recommendations to the user.

Optionally, the inertial measuring sensors, the photoplethysmogram sensor and the blood glucose sensor are located in a wearable user device.

According to one embodiment, the system further comprises a communication unit configured to transmit the generated recommendations to external devices.

The communication unit is further configured to communicate with weights to receive data on the user's weight and analyze changes in the user's weight over time and to analyze changes in the user's blood glucose level over the same period.

The glucose sensor is a non-invasive glucose sensor or an invasive glucose sensor.

Optionally, the storage module, the processing unit and the display are also located in the wearable user device.

Optionally, the storage module, the processing unit and the display are located in a separate smart device, wherein the system for providing recommendations for maintaining a healthy lifestyle basing on user's daily activity parameters further comprises a communication unit configured to transmit the measured user's daily activity parameters to the processing unit and the storage module.

Optionally, the system for providing recommendations for maintaining a healthy lifestyle basing on user's daily activity parameters further comprises a second storage module, a second processing unit and a second display located in a separate smart device, said system for providing recommendations for maintaining a healthy lifestyle basing on user's daily activity parameters further comprises the communication unit configured to transmit the measured parameters of user's daily activity also to the second processing unit and to the second storage module, and the second display is also configured to display data to the user.

Optionally, the system for providing recommendations for maintaining a healthy lifestyle basing on user's daily activity parameters comprises a GPS-receiver, configured to determine a user's current geolocation, and an additional processing unit configured to correct the results of estimation of the user's daily activity by said machine learning algorithm basing on geolocation data of the user.

According to a third aspect of the present invention, there is provided a method for providing recommendations for maintaining a healthy lifestyle basing on user's daily activity parameters automatically tracked in real time, comprising the steps of:
measuring automatically the user's daily activity parameters, including periods of physical activity, heart rate, the number of steps taken, the period of sleep time, changes in blood glucose, the amount of carbohydrates and calories taken with food;
training a machine learning algorithm to estimate the user's daily activity basing on the measured parameters of the user's daily activity and a predefined user profile containing the user's gender, age, height and weight;
generating recommendations for maintaining of the user's healthy lifestyle basing on estimation of the user's daily activity received as a result of using the machine learning algorithm; and
displaying the generated recommendations to the user.

According to another aspect of the present invention, there is provided a system for providing recommendations for maintaining a healthy lifestyle basing on user's daily activity parameters automatically tracked in real time, comprising:
inertial measuring sensors, including an accelerometer and a gyroscope;
a photoplethysmogram sensor;
a blood glucose sensor,

wherein the inertial measuring sensors, the photoplethysmogram sensor and the blood glucose sensor are configured to automatically measure the user's daily activity parameters, including periods of physical activity, heart rate, the number of steps taken, a sleep time period, changes in blood glucose level, the amount of carbohydrates and calories taken with food;
a processing unit configured to train a machine learning algorithm to estimate the user's daily activity basing on the measured parameters of the user's daily activity and a predefined user profile containing the user's gender, age, height and weight;
a storage module configured to store the predefined user profile, the measured parameters of the user's daily activity and estimation of the user's daily activity received as a result of using the machine learning algorithm,

wherein the processing unit is further configured to generate recommendations for maintaining of the user's healthy lifestyle basing on estimation of the user's daily activity, and the storage module is configured to store the generated recommendations,

wherein the system for providing recommendations for maintaining a healthy lifestyle basing on the user's daily activity parameters further comprises a display configured to display the generated recommendations to the user.

Optionally, the system for providing recommendations for maintaining a healthy lifestyle basing on user's daily activity parameters comprises a GPS-receiver, configured to determine a user's current geolocation and an additional processing unit, configured to correct the results of estimation of the user's daily activity by said machine learning algorithm basing on the geolocation data of the user.

According to the fifth aspect of the present invention there is provided a method for providing recommendations for maintaining a healthy lifestyle basing on user's daily activity parameters automatically tracked in real time, comprising the steps of:
measuring automatically the user's daily activity parameters, including periods of physical activity, heart rate, the number of steps taken, a period of sleep time, changes in blood glucose level, the amount of carbohydrates and calories taken with food;
determining indirectly the change in blood glucose level basing on the measured parameters of the user's daily activity, data on ambient sounds, geolocation, user schedules and user profiles containing the user's gender, age, height and weight;
training a machine learning algorithm to estimate the user's daily activity basing on the measured parameters of the user's daily activity, the determined change in blood glucose level and the predefined user profile;
generating recommendations for maintaining of user's healthy lifestyle basing on estimation of the user's daily activity received as a result of using the machine learning algorithm; and
displaying the generated recommendations to the user.

According to another aspect of the present invention, there is provided a system for providing recommendations for maintaining a healthy lifestyle basing on user's daily activity parameters automatically tracked in real time, comprising:
inertial measuring sensors, including an accelerometer and a gyroscope;
a photoplethysmogram sensor;

wherein the inertial measuring sensors and the photoplethysmogram sensor are configured to measure automatically the user's daily activity parameters, including periods of physical activity, heart rate, the number of steps taken, a sleep time period, the amount of carbohydrates and calories taken with food;
a microphone configured to record ambient sounds;
a GPS-receiver configured to determine a user's current geolocation;
an indirect glucose measurement unit configured to determine indirectly the changes in blood glucose level basing on the measured parameters of the user's daily activity, the data on ambient sounds, the geolocation, a predefined user schedule and a predefined user profile containing the user's gender, age, height and the weight;
a processing unit configured to train a machine learning algorithm to estimate the user's daily activity basing on the measured parameters of the user's daily activity, the determined change in blood glucose level and the predefined user profile;
a storage module configured to store the predefined user schedule, the predefined user profile, the measured parameters of the user's daily activity, the determined change in blood glucose level and estimation of the user's daily activity received as a result of using the machine learning algorithm,

wherein the processing unit is further configured to generate recommendations for maintaining of the user's healthy lifestyle basing on estimation of the user's daily activity, and the storage module is configured to store the generated recommendations,

wherein the system for providing recommendations for maintaining a healthy lifestyle basing on the user's daily activity parameters further comprises a display configured to display the generated recommendations to the user.

The technical result achieved by using the present invention is to provide real-time and user-independent tracking of user's daily activity parameters, including the change in the user's blood glucose level, followed by provision of recommendations for maintaining a healthy lifestyle to the user, generated basing on the machine learning algorithm trained by taking into account a physiological model of the user.

### Advantageous Effects of Invention

The technical result achieved by using the present invention is to provide real-time and user-independent tracking of user's daily activity parameters, including the change in the user's blood glucose level, followed by provision of recommendations for maintaining a healthy lifestyle to the user, generated basing on the machine learning algorithm trained by taking into account a physiological model of the user.

### BRIEF DESCRIPTION OF DRAWINGS

These and other features and advantages of the present invention will become apparent after reading the following description and viewing the accompanying drawings, in which:

FIG. 1 is a flowchart of a method for providing recommendations for maintaining a healthy lifestyle according to an embodiment of the present invention;

FIG. 2 represents a physiological model of glucose metabolism for the organism of a person suffering Type 1 diabetes;

FIG. 3(a) illustrates an exemplary graph of a change in a user's blood glucose level over time during a period of physical activity;

FIG. 3(b) illustrates an exemplary graph of a change in a user's blood glucose level over time during the period of experienced stress;

FIG. 4 is a flowchart for determining nutrition parameters of the user during the day according to one embodiment of the present invention;

FIG. 5 is an exemplary graph of correlation between the actual and the predicted number of calories taken by a plurality of users with food per day;

FIG. 6 shows a low-frequency trend of changes in blood glucose level that is not related to food intake, the resulting signal corresponding to the change in blood glucose level caused by the food intake, and the time moments at which the user began taking food are noted;

FIG. 7 illustrates: (701) a graph of likelihood of taking food by the user versus time, obtained using a machine learning algorithm according to one embodiment of the present invention; (703) a convolution graph with a normalized Gaussian kernel; (705) a graph of the result of processing the signal shown in graph (701) using a convolution with a normalized Gaussian kernel, shown in graph (703); (707) a resulting signal received after finding the local maximums of the signal shown in graph (705);

FIG. 8 shows the results of accuracy of a user's meals time estimated by the machine learning algorithm according to one embodiment of the present invention;

FIG. 9 is a graph of user's meals time estimation during the day basing on the user's blood glucose level and the recommended meals time;

FIG. 10 shows the results of accuracy of food intakes classification estimated by the machine learning algorithm according to one embodiment of the present invention.

The figures shown in the drawings serve to illustrate embodiments of the present invention only and are not intended limit it in any way.

### MODE FOR THE INVENTION

Various embodiments of the present invention are described in detail below with reference to the drawings. However, the present invention can be embodied in many other forms and should not be construed as being limited by any particular structure or function described in the following description. Basing on the present description, those skilled in the art will appreciate that the scope of legal protection of the present invention covers any embodiment of the present invention disclosed herein, regardless of whether it is implemented independently or in combination with any other embodiment of the present invention. For example, a system may be implemented or a method may be realized using any number of embodiments set forth herein. In addition, it should be understood that any embodiment of the present invention disclosed herein may be embodied using one or more elements of the claims.

The word “exemplary” is used herein to mean “serving as an example or illustration”. Any implementation described herein as “exemplary” need not be construed as being preferred or prevailing over other embodiments.

Currently, more and more people in the world are striving to lead a healthier lifestyle, trying to abandon the consumption of unhealthy foods in favor of a healthy and balanced food composition, are engaged in more active activities, observe the daily regimen. In particular, having chosen healthy and balanced food, people began to care about the amount of nutrients consumed: proteins, fats and carbohydrates. According to the present invention, an appropriate solution has been proposed that helps the user maintain his health and physical form. Namely, a method is proposed for automatic round-the-clock tracking of the user's daily activity, analysis of data received using the appropriate machine learning algorithm and providing recommendations to the user for maintaining a healthy lifestyle. In addition, a corresponding system has been proposed, comprising sensors for measuring parameters of the user's daily activity and a processing unit for processing these parameters and generating recommendations for implementing the aforementioned method.

According to the claimed invention, the user's daily activity parameters are periods of activity, the number of calories taken/wasted, heart rate, the number of steps taken, changes in blood glucose levels, sleep time, etc.

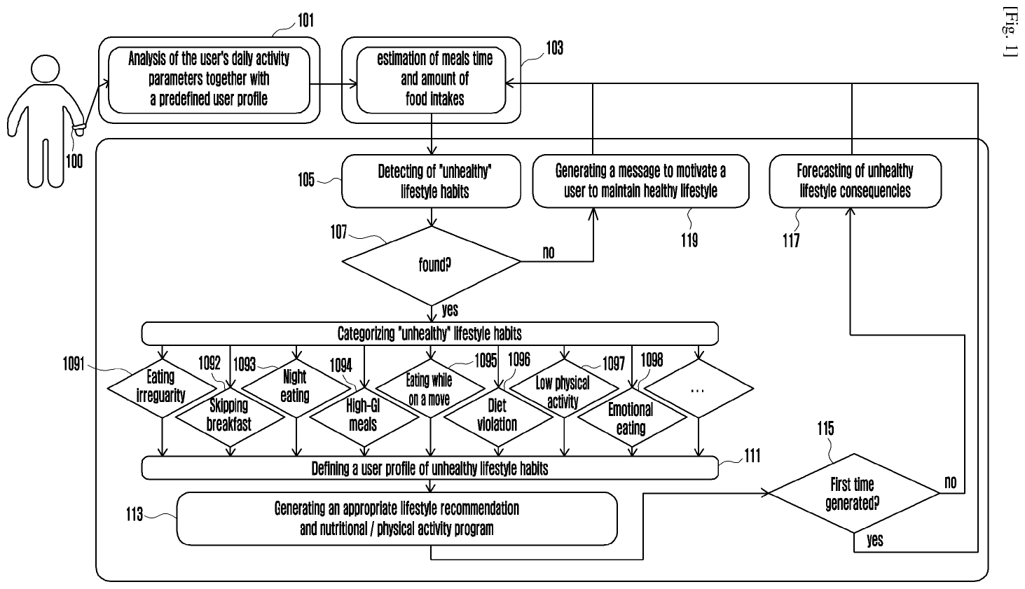

FIG. 1 shows a flowchart of a method for providing recommendations for maintaining a healthy lifestyle basing on user's daily activity parameters automatically tracked in real time, described herein.

In particular, it is assumed that the user has a wearable device 100, for example, a smart watch, a fitness bracelet, etc., which is configured to measure various parameters of the user's daily activity, i.e. containing appropriate sensors(e.g., sensor module) for measuring these parameters. Said system for providing recommendations for maintaining a healthy lifestyle basing on user's daily activity parameters comprises also a storage module(e.g., memory) that stores a predefined profile of a particular user, including biological characteristics of a person, such as gender, age, height, weight, etc. As said user's daily activity parameters are received, they are analyzed in the corresponding main processing unit(e.g., processor) together with a predefined user profile. Then, at operation 103, basing on the result of this analysis, the meals time and the amount of food taken by the user are estimated. If, as a result of the estimation, no actions or habits of the user are classified as healthy lifestyle ones, then a message is generated that motivates the user to continue to lead a healthy lifestyle. If unhealthy lifestyle habits are detected, these habits are correlated to categories of unhealthy habits, such as: eating irregularity, skipping breakfast, night eating, high glycemic index (GI) meals, eating while on a move, diet violation (dietary regimen), low physical activity, emotional overeating, insufficient sleep time, etc., wherein the categories of unhealthy habits are predefined and stored in the storage module. Further, the categories of unhealthy habits, with which the detected habits that were not conducive to maintaining a healthy lifestyle were correlated, are combined to form a personalized profile of unhealthy habits, which is used to further analysis and generation of an appropriate recommendation for a healthy lifestyle and a program regarding the user's nutrition and physical activity. In particular, when detecting emotional overeating, the system can track the user's stress level and inform him about the possible onset of emotional overeating while providing a corresponding recommendation motivating the user to engage in any type of activity, or a recommendation to contact the user's psychologist for consultation (or automatically connect with a psychologist if his contact number was previously stored by the user in said system). If a systematic intake of high carbohydrate foods by the user is detected, the system can generate informational messages for the user describing the benefits of low carbohydrate foods or recommend the user to contact his nutritionist or endocrinologist (or connect with a nutritionist or endocrinologist directly if their numbers are previously stored for communication in the system).

If such a recommendation for maintaining a healthy lifestyle and/or a program regarding nutrition and physical activity of a user is provided to the user for the first time, then the system proceeds again to the step of analyzing the user's daily activity parameters. If such a recommendation for maintaining a healthy lifestyle and/or a program regarding nutrition and physical activity of the user is provided to the user not for the first time, then a message is generated for the user notifying the user of possible bad health consequences caused by the detected unhealthy lifestyle habits, after that the system also proceeds to the step of estimating the meals time and the amount of food taken by the user, by taking into account information about recommendations provided to this user before (and therefore, by taking into account the eating habits of the user).

In addition, the user himself can set a goal to improve any of the daily activity parameters using the input means of the system for providing recommendations for maintaining a healthy lifestyle basing on user's daily activity parameters (buttons to select the corresponding item in the previously saved menu on a wearable device, mechanical or touch keyboard on a smart device of the system, etc.), for example, to reduce weight, to increase physical activity per day, to sleep more and etc. The system will generate recommendations to the user, motivating him to achieve his goal. This system can also be demanded by insurance companies that monitor implementation of the recommendations prescribed by doctor to their clients to regulate the conditions for the provision of insurance services. For example, if the patient-client of an insurance company fails to comply with the doctor's instructions, the client may be subsequently denied access/increased price when applying.

A wearable user device 100, comprising the necessary built-in sensors to measure the parameters of the user's daily activity, allow for receiving continuous data in real time. In addition, the presence of these built-in sensors allows receiving all the data necessary for analysis—the user's daily activity parameters, automatically, i.e. in user-independent mode. The user-independent mode is a mode of operation that does not require the user to input any data, all data is received automatically.

Thus, the system for providing recommendations for maintaining a healthy lifestyle basing on user's daily activity parameters automatically tracked in real time, comprises a set of sensors, preferably included in one portable user device, a storage module, a processing unit and a display. Optionally, the storage module, the processing unit, and the display can also be incorporated in a wearable user device 100, or can be incorporated in a separate smart device. According to another embodiment, the system for providing recommendations for maintaining a healthy lifestyle basing on user's daily activity parameters comprises two processing units, memory modules and displays, each being incorporated in a wearable user device 100 and a smart device.

A wearable user device 100 includes the following hardware modules: a communication unit, a device power control unit, a GPS-receiver, and a set of sensors containing inertial measuring sensors (accelerometer, gyroscope) and a photoplethysmogram sensor (PPG). In addition, according to one embodiment of the claimed invention, the wearable user device 100 further includes a blood glucose sensor. In addition, according to one embodiment of the claimed invention, the wearable user device 100 further includes a glucose sensor. Optionally, the user may have a plurality of wearable devices 100, each containing one or more sensors for measuring said parameters of the user's daily activity, the main thing is that the whole plurality of wearable devices include a device power control unit, said plurality of sensors and, optionally, a glucose sensor, and one of them necessarily includes, as indicated above, a GPS-receiver configured to determine the user's current geolocation, and a communication unit configured to receive data from all of the plurality of wearable devices, and an optional processing unit, an optional storage unit and an optional display in case they are incorporated in the wearable user device 100. Said glucose sensor may be any type of sensor capable of receiving information regarding a user's blood glucose level. In particular, it can be either an invasive sensor (a glucose sensor with an electrochemical sensor inserted under the skin, a sensor with an implantable part), or a non-invasive sensor (basing on an optical sensor—PPG sensor, a spectroscopic sensor; basing on the electric sensor (impedance spectroscopy), basing on several sensors). In addition, the system for providing recommendations for maintaining a healthy lifestyle basing on user's daily activity parameters automatically tracked in real time may additionally comprise an additional processing unit configured to correct the results of estimation of the user's daily activity by said machine learning algorithm basing on the user's geolocation data.

As an alternative embodiment, instead of a glucose sensor, an indirect glucose measurement unit can also be used for an indirect glucose measurement basing on PPG sensor data, data of inertial measuring sensors, data about ambient sounds (obtained using the corresponding microphone included in the considered system), a user profile, a geolocation, a user schedule, etc. Examples of invasive glucose sensors capable of monitoring continuously a user's blood glucose level are Medtronic iPro2, Dexcom G4/5, Abbott Freestyle Libre, etc. Examples of functioning of an indirect glucose measurement unit are receiving the user's geolocation data and determining that the user is in a restaurant, analyzing the user's schedule data, which indicates that the time the user visits the restaurant is the user's lunch time, receiving data on the user's movement and detecting hand movements specific to eating habits of the user, receiving data on ambient sounds and identifying sounds characteristic of the user's eating, etc. Regarding the use of the indirect glucose measurement unit instead of the glucose sensor, it is important to note that the hand on which the wearable user device 100 with this unit is worn will additionally affect the accuracy of the results of estimation of the user's daily activity parameters. In particular, the accuracy of estimation results with the wearable user device 100 worn on the prevailing hand (the one he eats with) will be slightly higher in comparison with the accuracy of estimation the results with a wearable user device 100 worn not on the prevailing hand. It will be apparent to those skilled in the art that the specific examples described above are merely illustrative and are not limited to the particular demonstrated variants of user's meal. The storage module of the system is also configured to register and store all measured user's daily activity parameters.

In particular, according to one embodiment, data of continuous monitoring of a user's blood glucose level is used to determine eating habits of a particular user. Namely, a processing unit receives data from said glucose sensor and from a plurality of sensors and calculates the following parameters: 1) meals times, 2) the number of meals per day, 3) the amount of carbohydrates in the food taken, 4) the number of calories taken by the user with food, basing on glucose change curves. Thus, if a wearable user device 100 has a processing unit, a storage module and a display, all calculations are made on the wearable user device 100 itself, and the results of the calculations and cor-responding recommendations can be displayed directly on the display of the wearable user device 100 itself and, if necessary, sent using a unit communication to any external devices.

If there is a processing unit, a storage module and a display in a smart device separate from the wearable user device 100, the processing unit receives data from said blood glucose sensor and the plurality of sensors using the communication unit, and the calculation results and corresponding recommendations can be displayed on said separate smart device.

According to another embodiment, the processing unit of the wearable user device 100 may receive data from the blood glucose sensor and the plurality of sensors for preliminary data processing, thereafter the communication unit transmits the preliminarily processed data to the processing unit of the smart device for final data processing, in particular for calculating the parameters 1)-4) and displaying the calculation results and the corresponding recommendations on the display of said separate smart device. According to this embodiment, the calculation results and the corresponding recommendations can also be transmitted back to the communication unit to display this data on the display of the wearable user device 100 as well.

Tracking the changes in the user's blood glucose level using said glucose sensor allow estimating the user's eating habits without the need for any actions on the part of the user (operation in a user-independent mode). In addition, the use of an accelerometer/gyroscope and a PPG sensor makes it possible to track efficiently the user's daily activity parameters (regarding nutrition, activity, sleep) in a user-independent mode.

In addition, an important advantage of the claimed invention is the use of a modified physiological model of the user, which is trained using the results of measuring the user's blood glucose level inputted therein and the output is a calculated individual response of the user to a particular food taken. The data on the individual response of the user's body is used as auxiliary data for training the machine learning algorithm used to estimate the meals time and classifying food intakes over one or several days. In particular, convolutional neural networks, recurrent neural networks as well as methods of mathematical statistics or other known methods of machine learning can be used as a machine learning algorithm. The measured user's daily activity parameters, including the results of measuring the blood glucose level of the user are inputted in such a machine learning algorithm for training it as well as auxiliary data for training, namely, data on the individual response of the user's body.

According to another possible embodiment, the system for providing recommendations for maintaining a healthy lifestyle basing on user's daily activity parameters is further configured to receive manually inputted data from the user regarding the user's daily activity parameters, for example, manually inputted names of the food taken or downloading photos of the food taken. In particular, the user can manually input the required parameters when using the device for the first time to specify the initial calibration of the computational physiological model for this particular user. The processing unit, in its turn, is configured to implement said algorithm, including the analysis of data inputted by the user (for example, determining the calorie content of food inputted by the user, or recognizing food in the user's photo and the subsequent determining its calorie content).

The estimation results of the meals time and classification of food intakes, obtained using the machine learning algorithm according to the present invention, are compared with the calibration result of the computational physiological model, and the comparison result is used to refine the estimation of nutrition parameters, i.e. the physiological model calculates the expected response to the amount of food calculated by the algorithm, this expected response is compared with the real response of the user's body and, if they diverge crudely, the algorithm recalculates the amount of food (training of the algorithm with real responses being accumulated over a certain period of time—from several hours to a few days). Thus, the accuracy of estimation of the meals time and classification of food intakes is improved, by taking into account a physiological model calibrated for a particular user. If necessary, the user can correct manually the estimated meals time and sleep time.

The traditional physiological model is a system of differential equations for concentrations or quantities of substances in various organs (liver, blood, intercellular fluid . . . ) of a human body when considering the kinetics of glucose absorption for the entire human body.

The electronic device 100 may be a user wearable device 100. The electronic device 100 may be the same as or similar to the wearable user device 100.

The electronic device 100 may include an inertial measurement sensor, a photoplethysmogram sensor, a glucose sensor, a processing unit (e.g., a processor), a storage module, a display and/or a GPS(global positioning system) device.

At operation 101, under the control of a processing unit(e.g., a processor), the electronic device 100 may receive the user's daily activity parameters by at least on sensor, and analyze the user's daily activity parameters together with a predefined user profile.

At operation 103, under the control of a processing unit(e.g., a processor), the electronic device 100 may estimate the meals time and the amount of food taken by the user based on the result of this analyzing the user's daily activity parameters.

At operation 105, under the control of a processing unit(e.g., a processor), the electronic device 100 may detect unhealthy lifestyle habits as a result of the estimation.

At operation 107, under the control of a processing unit(e.g., a processor), the electronic device 100 may determine whether an unhealthy lifestyle habit is found.

At operation 107, if it is determined that an unhealthy lifestyle habit is found, under the control of a processing unit(e.g., a processor), the electronic device 100 may proceed to operation 109.

At operation 107, if it is determined that an unhealthy lifestyle habit is not found, under the control of a processing unit(e.g., a processor), the electronic device 100 may proceed to operation 119.

As a result of the estimation, no actions or habits of the user are classified as healthy lifestyle ones, at operation 119, under the control of a processing unit(e.g., a processor), the electronic device 100 may generate a message that motivates the user to continue to lead a healthy lifestyle. The generated message may be displayed through the display of the electronic device 100.

If unhealthy lifestyle habits are detected, these habits are correlated to categories of unhealthy habits, such as: eating irregularity 1091, skipping breakfast 1092, night eating 1093, high glycemic index (GI) meals 1094, eating while on a move 1095, diet violation (dietary regimen) 1096, low physical activity 1097, emotional overeating 1098, insufficient sleep time, etc., at operation 109, under the control of a processing unit(e.g., a processor), the electronic device 100 may predefine the categories of unhealthy habits and store the categories of unhealthy habits in the storage module.

At operation 111, under the control of a processing unit(e.g., a processor), the electronic device 100 may combine the categories of unhealthy habits, with which the detected habits that were not conducive to maintaining a healthy lifestyle were correlated, to form a personalized profile of unhealthy habits, which is used to further analysis and generation of an appropriate recommendation for a healthy lifestyle and a program regarding the user's nutrition and physical activity.

At operation 113, under the control of a processing unit(e.g., a processor), the electronic device 100 may further analyze and/or generate an appropriate recommendation for the healthy lifestyle and the program regarding the user's nutrition and physical activity based on the personalized profile of unhealthy habits.

when detecting emotional overeating, at operation 113, under the control of a processing unit(e.g., a processor), the electronic device 100 can track the user's stress level and inform him about the possible onset of emotional overeating while providing a corresponding recommendation motivating the user to engage in any type of activity, or a recommendation to contact the user's psychologist for consultation (or automatically connect with a psychologist if his contact number was previously stored by the user in said system).

If a systematic intake of high carbohydrate foods by the user is detected, the system can generate informational messages for the user describing the benefits of low carbohydrate foods or recommend the user to contact his nutritionist or endocrinologist (or connect with a nutritionist or endocrinologist directly if their numbers are previously stored for communication in the system).

At operation 115, under the control of a processing unit(e.g., a processor), the electronic device 100 may determine whether a recommendation for maintaining a healthy lifestyle and/or a program regarding nutrition and physical activity of a user is provided to the user for the first time.

At operation 115, under the control of a processing unit(e.g., a processor), the electronic device 100 determines the recommendation for maintaining a healthy lifestyle and/or a program regarding nutritional and physical activity of the user is provided to the user for the first time, the electronic device 100 may proceed to operation 103.

At operation 115, under the control of a processing unit(e.g., a processor), the electronic device 100 determines the recommendation for maintaining a healthy lifestyle and/or a program regarding nutrition and physical activity of the user was not provided to the user for the first time, the electronic device 100 may proceed to operation 117.

if such a recommendation for maintaining a healthy lifestyle and/or a program regarding nutrition and physical activity of a user is provided to the user for the first time, under the control of a processing unit(e.g., a processor), the electronic device 100 proceeds again to the step of analyzing the user's daily activity parameters.

If such a recommendation for maintaining a healthy lifestyle and/or a program regarding nutrition and physical activity of the user is provided to the user not for the first time, under the control of a processing unit(e.g., a processor), the electronic device 100 may generate a message for the user notifying the user of possible bad health consequences caused by the detected unhealthy lifestyle habits, after that the system also proceeds to the step of estimating the meals time and the amount of food taken by the user, by taking into account information about recommendations provided to this user before (and therefore, by taking into account the eating habits of the user).

At operation 117, under the control of a processing unit(e.g., a processor), the electronic device 100 may generate a message for the user notifying the user of possible bad health consequences caused by the detected unhealthy lifestyle habits, after that the electronic device 100 also proceeds to the step of estimating the meals time and the amount of food taken by the user, by taking into account information about recommendations provided to this user before (and therefore, by taking into account the eating habits of the user).

At operation 101, under the control of a processing unit(e.g., a processor), the system may receive the user's daily activity parameters by at least on sensor, and analyze the user's daily activity parameters together with a predefined user profile.

At operation 103, under the control of a processing unit(e.g., a processor), the system may estimate the meals time and the amount of food taken by the user based on the result of this analyzing the user's daily activity parameters.

At operation 105, under the control of a processing unit(e.g., a processor), the system may detect unhealthy lifestyle habits as a result of the estimation.

At operation 107, under the control of a processing unit(e.g., a processor), the system may determine whether an unhealthy lifestyle habit is found.

At operation 107, if it is determined that an unhealthy lifestyle habit is found, under the control of a processing unit(e.g., a processor), the system may proceed to operation 109.

At operation 107, if it is determined that an unhealthy lifestyle habit is not found, under the control of a processing unit(e.g., a processor), the system may proceed to operation 119.

As a result of the estimation, no actions or habits of the user are classified as healthy lifestyle ones, at operation 119, under the control of a processing unit(e.g., a processor), the system may generate a message that motivates the user to continue to lead a healthy lifestyle. The generated message may be displayed through the display of the system.

If unhealthy lifestyle habits are detected, these habits are correlated to categories of unhealthy habits, such as: eating irregularity 1091, skipping breakfast 1092, night eating 1093, high glycemic index (GI) meals 1094, eating while on a move 1095, diet violation (dietary regimen) 1096, low physical activity 1097, emotional overeating 1098, insufficient sleep time, etc., at operation 109, under the control of a processing unit(e.g., a processor), the system may predefine the categories of unhealthy habits and store the categories of unhealthy habits in the storage module.

At operation 111, under the control of a processing unit(e.g., a processor), the system may combine the categories of unhealthy habits, with which the detected habits that were not conducive to maintaining a healthy lifestyle were correlated, to form a personalized profile of unhealthy habits, which is used to further analysis and generation of an appropriate recommendation for a healthy lifestyle and a program regarding the user's nutrition and physical activity.

At operation 113, under the control of a processing unit(e.g., a processor), the system may further analyze and/or generate an appropriate recommendation for the healthy lifestyle and the program regarding the user's nutrition and physical activity based on the personalized profile of unhealthy habits.

when detecting emotional overeating, at operation 113, under the control of a processing unit(e.g., a processor), the system can track the user's stress level and inform him about the possible onset of emotional overeating while providing a corresponding recommendation motivating the user to engage in any type of activity, or a recommendation to contact the user's psychologist for consultation (or automatically connect with a psychologist if his contact number was previously stored by the user in said system).

If a systematic intake of high carbohydrate foods by the user is detected, the system can generate informational messages for the user describing the benefits of low carbohydrate foods or recommend the user to contact his nutritionist or endocrinologist (or connect with a nutritionist or endocrinologist directly if their numbers are previously stored for communication in the system).

At operation 115, under the control of a processing unit(e.g., a processor), the system may determine whether a recommendation for maintaining a healthy lifestyle and/or a program regarding nutrition and physical activity of a user is provided to the user for the first time.

At operation 115, under the control of a processing unit(e.g., a processor), the system determines the recommendation for maintaining a healthy lifestyle and/or a program regarding nutritional and physical activity of the user is provided to the user for the first time, the system may proceed to operation 103.

At operation 115, under the control of a processing unit(e.g., a processor), the system determines the recommendation for maintaining a healthy lifestyle and/or a program regarding nutrition and physical activity of the user was not provided to the user for the first time, the system may proceed to operation 117.

if such a recommendation for maintaining a healthy lifestyle and/or a program regarding nutrition and physical activity of a user is provided to the user for the first time, under the control of a processing unit(e.g., a processor), the system proceeds again to the step of analyzing the user's daily activity parameters.

If such a recommendation for maintaining a healthy lifestyle and/or a program regarding nutrition and physical activity of the user is provided to the user not for the first time, under the control of a processing unit(e.g., a processor), the system may generate a message for the user notifying the user of possible bad health consequences caused by the detected unhealthy lifestyle habits, after that the system also proceeds to the step of estimating the meals time and the amount of food taken by the user, by taking into account information about recommendations provided to this user before (and therefore, by taking into account the eating habits of the user).

At operation 117, under the control of a processing unit(e.g., a processor), the system may generate a message for the user notifying the user of possible bad health consequences caused by the detected unhealthy lifestyle habits, after that the system also proceeds to the step of estimating the meals time and the amount of food taken by the user, by taking into account information about recommendations provided to this user before (and therefore, by taking into account the eating habits of the user).

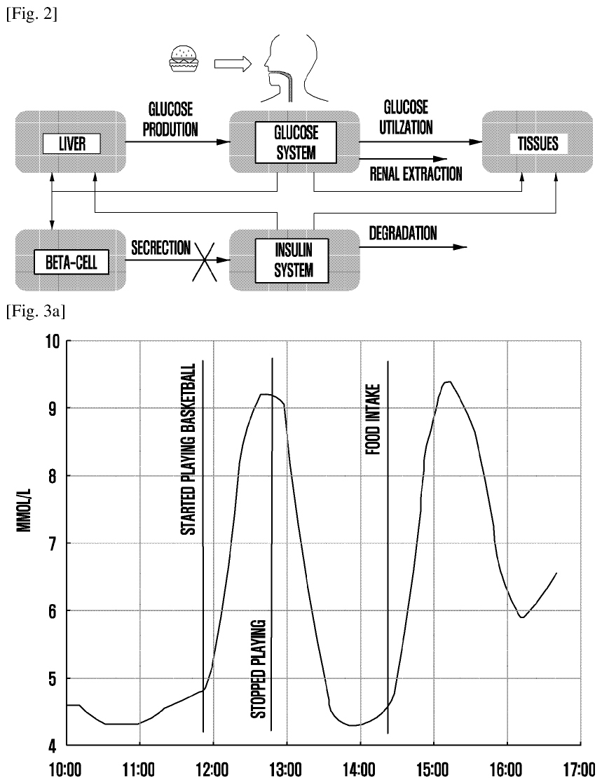

FIG. 2 presents a physiological model when considering the kinetics of glucose absorption for the organism of a person suffering Type 1 diabetes, in accordance with the traditional method, which models the distribution and dynamic changes in the concentration of glucose and insulin in various organs and tissues using available experimental data. In particular, FIG. 2 shows a physiological model for people suffering Type 1 diabetes, which depicts a glucose metabolism system that is formed by production of glucose by the liver and intake of food containing glucose, said blood glucose level is maintained by an insulin regulation system that is formed by the administration of insulin in a person suffering Type 1 diabetes. This physiological model takes into account the uptake of glucose by tissues, renal extraction of glucose, as well as insulin entry into the bloodstream and destruction of insulin, here, glucose and insulin conversions are shown by bold arrows on the figure, and the corresponding control signals are shown by thin arrows (see Dalla Man C., Breton M., Cobelli C.—“Physical Activity into the Meal Glucose—Insulin Model of Type 1 Diabetes: In Silico Studies”, Parker, R. S., Doyle, F. J., & Peppas, N. A. ? “A model-based algorithm for blood glucose control in Type I diabetic patient” or Sveshnikova A. N., Panteleev M. A., Dreval A. V., Shestakova T. P., Medvedev O. S., Dreval O. A.—“Theoretical estimation of glucose metabolism parameters basing on continuous glycemia monitoring data using mathematical modeling”). As illustrated in FIG. 2, patients suffering Type 1 diabetes do not have their own secretion of insulin, so the arrow responsible for insulin secretion is depicted crossed out. This known traditional method is effectively applicable to description of reactions of physiological parameters of patients suffering Type 1 diabetes to food, but is difficult for healthy users due to the presence of normal insulin secretion, which complicates the system of differential equations (more variables in the equations).

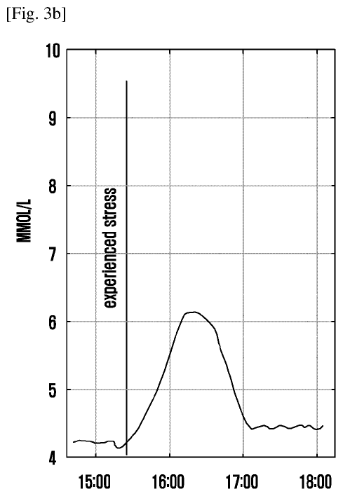

According to the present invention, a modified physiological model is used, that takes into account the intrinsic insulin secretion of a user, and in addition, takes into account both physical activity, heart rate, and stress. In addition, according to the considered modified physiological model, daily changes in glucose persistence (“glucose tolerance”) are also taken into account, and the model itself is used to calibrate the parameters of food taken. The traditional model does not take into account any of the above factors. These factors are extremely important when analyzing the user's daily activity parameters, since the blood glucose level of the user depends not only on food taken, but also on stress and on intense physical activity. Both stress and intense physical activity give rise to a response in blood glucose level, similar to the response obtained as a result of taking food. For the example, FIG. 3(a) and FIG. 3(b) show the corresponding graphs of a change in a user's blood glucose level over time during basketball and during a period of experienced stress, respectively. As illustrated in FIG. 3(a), when a user started a basketball lesson (physical activity), the glucose level in his blood began to rise and reached its peak value at the time the lesson stopped. Further, the user took food, thereafter his blood glucose level was increased as well. A similar picture is depicted in FIG. 3(b), albeit less marked one, which clearly shows that the blood glucose level of the user also increased after the stress experienced. The method for providing recommendations for maintaining a healthy lifestyle basing on user's daily activity parameters automatically tracked in real time, according to the present invention is intended to distinguish ongoing types of daily activities of the user with a high degree of accuracy compared to prior art solutions, thanks to a comprehensive analysis of the user's daily activity parameters. In addition, the metabolism in the body of each person is individual, therefore, each person reacts to the same food individually. Said modified physiological model, used in the present invention, calculates the individual response of the user's body to the amount of food taken by the user as calculated by the machine learning algorithm in a user-in-dependent mode after being training on the measured parameters of the user's daily activity. These calculated responses are then used as auxiliary data for further training of the machine learning algorithm.

In particular, as indicated in the “Background” section, the prior art solutions require often repeated manual input of information from the user, namely, the names of the food taken, therefore, the accuracy of the analysis of data associated with the food taken depends directly on the user's memory, his honesty and motivation. In addition, the known solutions do not consider the individual physiological characteristics of the user, therefore, the used calculation of the energy balance is the same for each user. As mentioned above, according to the discussed method, the user is not required to perform any routine actions, and all meals are recorded automatically by taking into account the relationship between nutrition, physical activity, sleep and individual characteristics of the glycemic response of the organism of a particular user. Thus, the discussed method reduces the percentage of errors in the recording of meals, improves the accuracy of classification of food intakes in a user-independent mode. In addition, this method is compatible with some available wellness tracking apps, for example, the Samsung Health app.

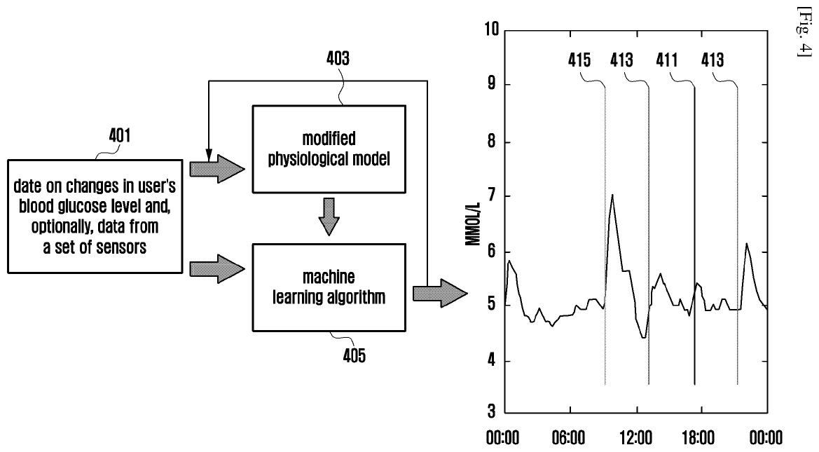

FIG. 4 is a flowchart for determining nutrition parameters of the user during the day according to one embodiment of the present invention. At operation 401, the processing unit receives data on changes in the blood glucose level of the user, measured continuously during the day, from the glucose sensor. At operation 403, this data and data from the mentioned plurality of sensors are inputted to a modified physiological model for calibrating its parameters. At operation 405, data on changes in the user's blood glucose level and, optionally, data from said plurality of sensors is also inputted to the machine learning algorithm, which will be described in more detail below. Then, the parameters of the modified physiological model of the user, calibrated basing on changes in glucose level and data from the plurality of sensors, are also inputted to the algorithm for its further training. According to another embodiment, only parameters of the modified physiological model of the user calibrated basing on changes in the glucose level and data from the plurality of sensors are inputted to the algorithm. As a result of using this algorithm, the user's meals time during the day and the amount of food taken by the user? the amount of carbohydrates in that food are estimated (calorie content can be estimated as well). In particular, in FIG. 4 first lines 411 indicate the estimated time of taking low carbohydrate food by a user, second lines 413—time of taking a mean carbohydrate food, and third lines 415—time of taking a high carbohydrate food. The estimated amount of food taken by the user, outputted by the said machine learning algorithm, is inputted also to the modified physiological model of the user to determine the response of the user's body to the amount of food products of each user calculated by the machine learning algorithm, as described above.

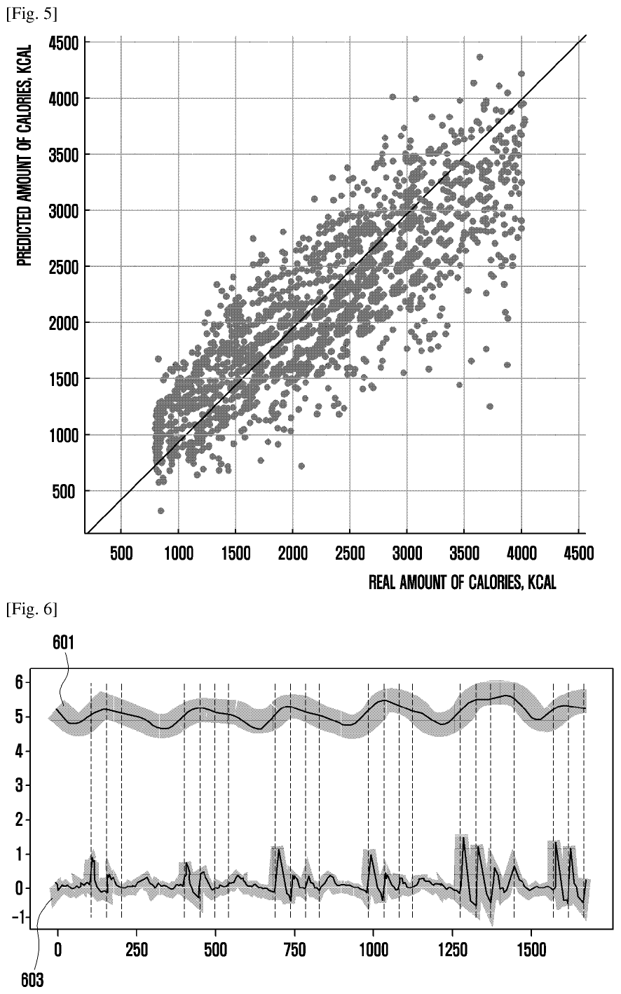

Basing on the estimated amount of carbohydrates and information from the user profile, the number of calories taken by the user with food is determined. FIG. 5 shows a graph of correlation between the actual and predicted number of calories taken by a plurality of users with food per day (NHANES WWEIA database was used). In particular, said plurality of users includes 2281 people. The X axis is the actual number of calories taken by a user with food per day, and the Y axis is the amount of calories predicted by the algorithm basing on data on the amount of carbohydrates and data on a predefined user profile (i.e., data on age, gender, weight and height of the user). Therefore, due to estimated number of calories taken and available data on the user's physical activity, the present invention can estimate the user's energy balance, which is an important characteristic of a healthy lifestyle, and generate a further recommendation for maintaining a healthy lifestyle basing on this estimated energy balance of the user.

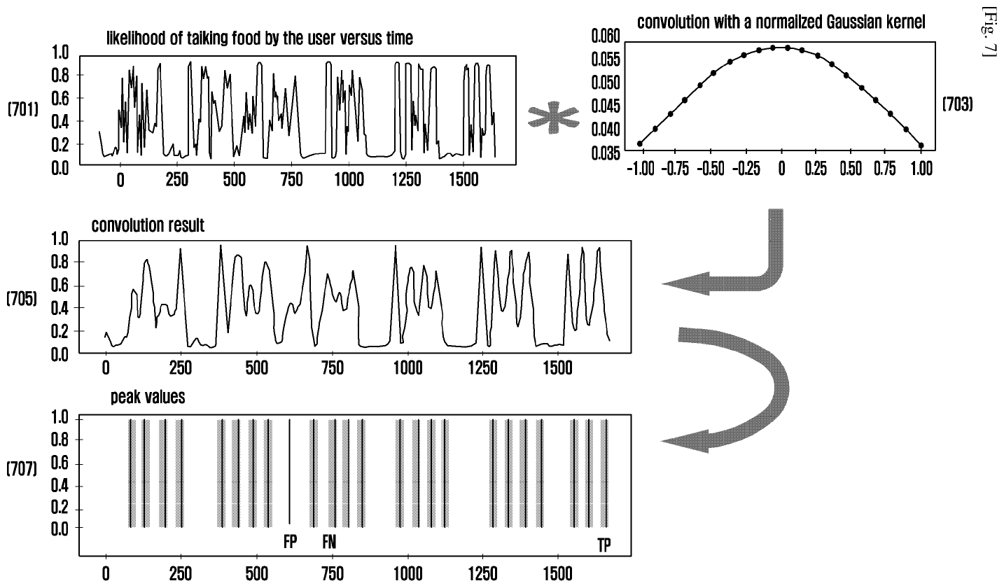

Further FIG. 6 shows a low-frequency trend of changes in blood glucose level that is not related to food intake (designated in 601 in the graph), the resulting signal corresponding to the change in blood glucose level caused by the a food intake (designated in 603 in the graph) and the time moments at which the user began taking food (designated in dot lines in the graph). The horizontal axis indicates the number of counts of the glucose sensor (in this example, one count corresponds to 5 minutes), and the vertical axis shows the glucose concentration in mmol/L. In particular, using a low-frequency digital filter (for example, a Butterworth filter with a cut-off frequency corresponding to a 12-hour period), a low-frequency trend of changes in a blood glucose level that is not related to food intake is designated (a response to a food intake corresponds to frequencies higher than the selected cut-off frequency filter). This low-frequency trend is then subtracted from the original signal of blood glucose level change to obtain a resulting signal, the original signal being a glucose change signal received from the glucose sensor. The resulting signal is characterized by relatively rapid changes in glucose dynamics corresponding to the response to food intakes. The peak values of the resulting signal are regarded as approximate time moments of beginning of taking food by the user, which are also marked in FIG. 6.

Next, the resulting signal is converted into a form convenient for the machine learning algorithm, as described below. For example, according to one embodiment, the signal is sampled with a sampling period of 5 minutes, thereafter the sampled signal is divided into windows (segments) of 2 hours, the windows intersecting each other with a shift increment of 5 minutes. In addition to the glucose change signal itself, additional features that improve the training quality can be added to the feature vector inputted to the machine learning algorithm, for example, such as PPG sensor data, inertial measuring sensor data, a sleep fraction, several orders of magnitude derivatives, and statistical characteristics of the signal in 2 hour window.

To estimate the meals time, each feature vector is classified by a trained machine learning algorithm (for example, the Random Forest algorithm with optimized parameters) in accordance with 2 classes: a “No food” class, which refers to the time period when the user did not take food, and a class “Food”, which refers to the period of time when the user took food. As a rule, the result of data classification by machine learning algorithm is quite “noisy”, i.e. there are many single erroneous results. Such errors can be eliminated by filtering the high frequency oscillations of the results, for example, FIG. 7 illustrates <701> a graph of likelihood of taking food by the user versus time, obtained using a machine learning algorithm according to one embodiment of the present invention. The horizontal axis shows the numbers of above mentioned windows with a shift increment of 5 minutes, count of the glucose sensor (in this example, one count corresponds to 5 minutes), and the vertical axis shows the likelihood of beginning taking food by the user in the corresponding window, obtained by the machine learning algorithm. Next, the estimated signal of likelihood of beginning taking food by the user in the corresponding window is filtered using some convolution. In particular, for example, in the present embodiment, a convolution with a normalized Gaussian kernel (for example, ?=1, ?2=1) is applied to this estimated signal, the graph of which is depicted by the letter <703> in FIG. 7. In addition, in FIG. 7, the letter <705> also shows a graph of the result of processing the signal shown in graph <701>, using a convolution with a normalized Gaussian kernel, shown in graph <703>, each peak value being regarded as a predicted meals time period. Therefore, after finding the local maximums of the signal shown on the graph <705>, the resulting signal is received <70>, which represents the predicted periods of meals time. In addition, the graph of the resulting signal also shows a certain neighborhood (designated in orange on the graph), indicating the time interval that admits a prediction error, for example, [−15 min; +30 min] relative to the beginning of taking food. Thus, it is possible to identify periods of time with the highest likelihood of taking food by the user, and the beginning of the corresponding time period can be regarded as the beginning of taking food by the user.

To test the efficiency of the algorithm, the number of true meal determinations (true positive, TP), false meal determinations (false positive, FP) and false meal omissions (false negative, FN) are calculated. As a rule, the efficiency of classification algorithms is estimated using an F1 measure (F1 score), which uses Precision and Recall as the basis:

$$F 1 score = {2}^{*} \frac{{Precision}^{*} Recall}{Precision + Recall} , \\ where$$

$$Precision = \frac{T P}{T P + F P} , \\ and$$

$$Recall = \frac{T P}{T P + F N} .$$

To estimate the amount of carbohydrates taken with food, only feature vectors classified as “Food” are used. Each of these feature vectors is estimated by a trained machine learning algorithm (for example, logistic regression with optimized parameters) in accordance with 3 classes: “Low” refers to the time period when the user took low-carbohydrate food (up to 49 grams), “Mid”—to the time period when the user took average-carbohydrate food (from 50 to 119 grams), “High”—to the time period when the user took high-carbohydrate food (from 120 grams).

Thus, the present invention allows for estimation of the amount of carbohydrates taken with food. Basing on the amount of carbohydrates and a predefined user profile, the excess of calories taken over calories consumed (overeating) can be estimated.

Upon completion of estimation of the meals time and classification of food intakes, taking into account a computational physiological model calibrated for a particular user, the method for providing recommendations for maintaining a healthy lifestyle basing on user's daily activity parameters, automatically tracked in real time, generates appropriate recommendations for the user. Unlike the known solutions that generally recommend that all users consume less-calorie foods or move more and do not monitor regularly the user's current food intakes (and individual glycemic response, respectively), the considered method offers effective individualized recommendations and/or an individual program for development of a healthy lifestyle of the user: this program uses both the results of the analysis of the user's daily activity parameters, as described above, and the analysis of the daily activity parameters of a plurality of other users of the considered system.

As was indicated above with respect to FIG. 1, if the result of the analysis has shown that no user habits are classified as unhealthy lifestyle ones, then a message is generated that motivates the user to continue to lead a healthy lifestyle. If unhealthy lifestyle habits are detected, then the considered method for providing recommendations for maintaining a healthy lifestyle basing on user's daily activity parameters, automatically tracked in real time, gives appropriate recommendations for development of a healthy lifestyle to the user. For example, if insufficient physical activity is detected, the considered method can generate a recommendation for a gradual increase in physical activity, with respect to a short sleep time the method may recommend going to bed earlier, if an irregular food intake is detected, the method may recommend a diet, etc. Thus, recommendations may include both advices on healthy and unhealthy diets, advices on an individual dietary regimen, and provision of an individual program on physical activity and sleep. The considered method can generate both daily recommendations based on the results of the measured parameters of the current day by taking into account the goals set for that day, and recommendations based on the analysis of parameters measured over a given period of time (week, month, etc.). If the user does not follow the recommendations, the method can additionally generate an information message notifying the user about the possible negative consequences of such a lifestyle. In addition, the considered system is configured to automatically check the compliance of the user's daily activity parameters with the recommendations previously provided by the system to motivate the user to continue to maintain a healthy lifestyle or warn the user about possible consequences of in-compliance with the provided recommendations, as indicated above.

According to another embodiment, the data from the blood glucose sensor and from said set of sensors is inputted directly to the above algorithm for further processing. In this embodiment, the modified physiological model for calculation of the individual response of the user's body is not used, which results in a decrease in accuracy of determining the time periods of taking food by the user as compared to the method in which such a modified physiological model is used, however, the accuracy of determining these time periods in comparison with the known solutions is still high.

According to another embodiment, the user can also periodically measure his weight using weights, here a wearable user device 100 equipped with a glucose sensor is configured to receive data from said weights and analyze changes in the user's weight over time and analyze changes in blood glucose level of the user over the same period, which improves accuracy in calibrating the corresponding computational physiological model of the user.

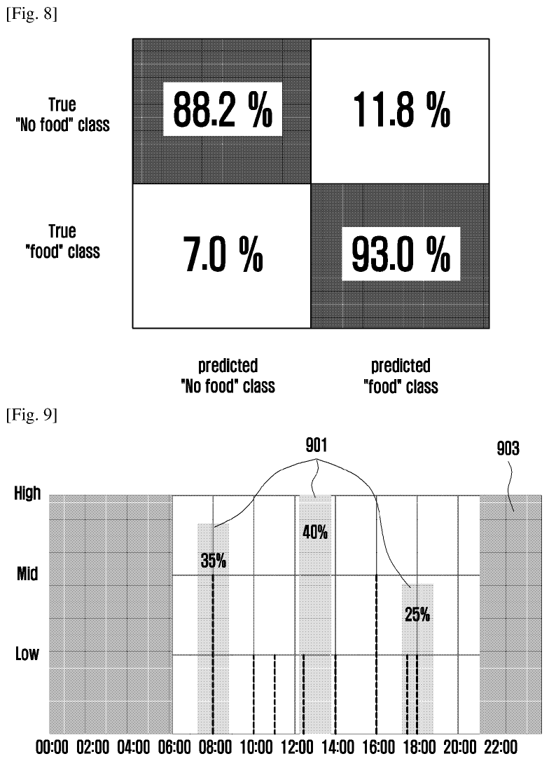

According to another embodiment, the processing unit of the claimed system is further configured to make long-term predictions regarding the user's condition in the current lifestyle of the user. In particular, such long-term predictions include prediction of future weight, prediction of life expectancy, etc. In addition, when a lifestyle changes by the user, the processing unit can also generate motivating messages for the user, for example, when improving the user's daily activity parameters, the processing unit can generate a motivating message that, according to the updated prediction of future weight, the user will lose weight to the desired weight, and if the user's daily activity parameters deteriorate, the processing unit can generate a motivating message that according to the updated prediction of the future weight, it is expected that the user will gain weight. To test the considered system for providing recommendations for maintaining a healthy lifestyle basing on user's daily activity parameters automatically tracked in real time, 50 volunteers were selected, who were equipped with the wearable user devices 100 described above, which measured the user's daily activity parameters for each of the selected volunteers (more than 1000 meals in total in the collected database). In particular, the blood glucose level of the user, data on the user's movements, data on the user's pulse, and data on sleep were measured. The claimed machine learning algorithm described above was trained basing on these measured data for estimation of a meals time and classification of food intakes. To test the efficiency of the algorithm, a cross-validation method was used with exclusion (data of one volunteer was excluded from the database, the algorithm was trained on the remaining data and then tested on the excluded data, the test results were averaged over all volunteers). FIG. 8 shows the results of accuracy of a user's meals time estimated by said algorithm with respect to the 50 volunteers. In particular, the accuracy of the meals time estimated by the algorithm, as compared to the actual meals time by the user, was 93% (percentage of error, respectively, was 7%), and the accuracy of estimated time during which the user did not take food was 88.2% (percentage of error, respectively, was 11.8%). Accordingly, the overall accuracy of the algorithm for estimation of a user's meals time was 90.43% (F1 measure=0.89). FIG. 9 is a graph of user's meals time estimation during the day basing on the user's blood glucose level and the recommended meals time for one of the volunteers mentioned above. According to a predefined profile of this volunteer, he was 54 years old, gender-female, weight-71 kg, height-149 cm. The activity level of this volunteer by the scale from 1 to 5 was defined as 1, and calculation of the energy spent showed 1578 kcal. Therefore, the algorithm determined that the volunteer had a normal energy balance and he took average-carbohydrate food. Basing on this data, a recommendation was provided to the user, in particular, the graph depicted in FIG. 9, in which first bars 901 indicate meals times that are recommended for this user, basing on observations, and second bars(designated in dot lines in the graph) indicate meals times determined by the above algorithm. The height of the columns related to the second bars in FIG. 9 corresponds to the class of food according to the amount of carbohydrates determined by the above algorithm. The time period recommended for the user's sleep is indicated with third bars 905 on the graph.

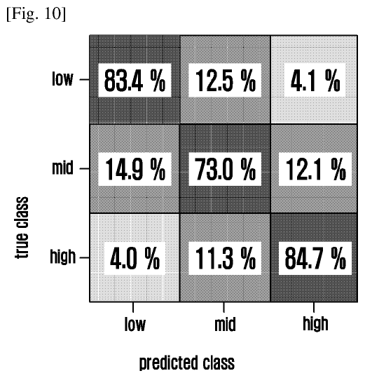

Further FIG. 10 shows the results of accuracy of food intakes classification estimated by said algorithm for the above-mentioned 50 volunteers. As can be seen in the figure, the accuracy of determining of taking low-carbohydrate food by the user as compared to his actual food intake was 83.4% (the error was 16.8% respectively), the accuracy of determining of taking average-carbohydrate food by the user as compared to his actual food intake was 73.0% (the error was 27% respectively) and the accuracy of determining of taking high-carbohydrate food by the user as compared to his actual food intake was 84.7% (the error was 15.3% respectively). Accordingly, the overall accuracy of the food classification algorithm was 80.4% (F1-measure=0.80).

Those skilled in the art would appreciate that, as necessary, the number of structural elements or components of the system can vary. The scope of protection of the present invention is intended to cover all possible different locations of the above structural elements of the system. In one or more exemplary embodiments, the functions described herein may be implemented in hardware, software, firmware, or any combination thereof. Being implemented in software, said functions may be stored on or transmitted in the form of one or more instructions or a code on a computer-readable medium. Machine-readable media include any storage medium that enables the transfer of a computer program from one place to another. A storage medium may be any available medium that is accessed by a computer. By way of example, but not limitation, such computer-readable media can be RAM, ROM, EEPROM, CD-ROM or other optical disk drive, magnetic disk drive or other magnetic storage devices, or any other storage medium that can be used for transfer or storage of the required program code in the form of instructions or data structures and which can be accessed using a computer. In addition, if the software is transferred from a website, server, or other remote source using coaxial cables, fiber optic cables, twisted pair, digital subscriber line (DSL), or using wireless technologies such as infrared, radio, and microwave, such wired and wireless means fall within the definition of media. Combinations of the aforementioned storage media should also fall within the protection scope of the present invention.

Although exemplary embodiments of the invention are shown in the present description, it should be understood that various changes and modifications can be made without departing from the scope of protection of the present invention defined by the attached claims. The functions, steps, and/or actions referred to in the claims characterizing the method in accordance with the embodiments of the present invention described herein need not be performed in any particular order unless otherwise noted or specified. Moreover, indication of elements of the system in the singular does not exclude a plurality of such elements, unless explicitly stated otherwise.

1. A method for providing recommendations for maintaining a healthy lifestyle basing on user's daily activity parameters automatically tracked in real time, comprising the steps of:
measuring automatically the user's daily activity parameters, including periods of physical activity, changes in blood glucose level, and data of a food intake;
building a physiological model basing on the measured change in the user's blood glucose level to determine an individual response of the user to a food intake;
training a machine learning algorithm to estimate the user's daily activity basing on the measured parameters of the user's daily activity, the determined individual response of the user and a predefined user profile containing the user's gender, age, height and weight;
generating recommendations for maintaining of the user's healthy lifestyle basing on estimation of the user's daily activity received as a result of using the machine learning algorithm; and
displaying generated recommendations to the user.

2. A system for providing recommendations for maintaining a healthy lifestyle basing on user's daily activity parameters automatically tracked in real time, comprising:
inertial measuring sensors, including an accelerometer and a gyroscope;
a photoplethysmogram sensor;
a blood glucose sensor,
wherein the inertial measuring sensors, the photoplethysmogram sensor and the blood glucose sensor are configured to automatically measure the user's daily activity parameters, including periods of physical activity, changes in blood glucose level, and data of a food intake;
a processing unit configured to build a physiological model basing on a change in the user's blood glucose level to determine an individual response of the user to a food intake and training a machine learning algorithm to estimate the user's daily activity basing on the measured parameters of the user's daily activity, the determined individual response of the user, and a predefined user profile containing the user's gender, age, height and weight;
a storage module configured to store the predefined user profile, the measured parameters of the user's daily activity, the determined individual response of the user and estimation of the user's daily activity received as a result of using the machine learning algorithm,
wherein the processing unit is additionally configured to generate recommendations for maintaining of the user's a healthy lifestyle basing on estimation of the user's daily activity, and the storage module is configured to store the generated recommendations,
wherein the system for providing recommendations for maintaining a healthy lifestyle basing on the user's daily activity parameters further comprises a display configured to display the generated recommendations to the user.

3. The system for providing recommendations for maintaining a healthy lifestyle basing on user's daily activity parameters according to claim 2,
wherein the inertial measuring sensors, the photoplethysmogram sensor and the blood glucose sensor are located in a wearable user device.

4. The system for providing recommendations for maintaining a healthy lifestyle basing on user's daily activity parameters according to claim 2, further comprising a communication unit configured to transmit the generated recommendations to external devices.

5. The system for providing recommendations for maintaining a healthy lifestyle basing on user's daily activity parameters according to claim 4,
wherein the communication unit is further configured to communicate with weights to receive data on the user's weight and analyze changes in the user's weight over time and to analyze changes in the user's blood glucose level over the same period.

6. The system for providing recommendations for maintaining a healthy lifestyle basing on user's daily activity parameters according to claim 2,
wherein the glucose sensor is a non-invasive glucose sensor or an invasive glucose sensor.

7. The system for providing recommendations for maintaining a healthy lifestyle basing on user's daily activity parameters according to claim 3,
wherein the storage module, the processing unit and the display are also located in the wearable user device.

8. The system for providing recommendations for maintaining a healthy lifestyle basing on user's daily activity parameters according to claim 3,
wherein the storage module, the processing unit and the display are located in a separate smart device, wherein the system for providing recommendations for maintaining a healthy lifestyle basing on user's daily activity parameters further comprises a communication unit configured to transmit the measured user's daily activity parameters to the processing unit and the storage module.

9. The system for providing recommendations for maintaining a healthy lifestyle basing on user's daily activity parameters according to claim 7, further comprising a second storage module, a second processing unit and a second display located in a separate smart device,
wherein said system for providing recommendations for maintaining a healthy lifestyle basing on user's daily activity parameters further comprises the communication unit configured to transmit the measured parameters of user's daily activity also to the second processing unit and to the second storage module, and the second display is also configured to display data to the user.

10. The system for providing recommendations for maintaining a healthy lifestyle basing on user's daily activity parameters according to claim 2, further comprising a GPS-receiver, configured to determine a user's current geolocation and an additional processing unit, configured to correct the results of estimation of the user's daily activity by said machine learning algorithm basing on geolocation data of the user.

11. A method for providing recommendations for maintaining a healthy lifestyle basing on user's daily activity parameters automatically tracked in real time, comprising the steps of:
measuring automatically the user's daily activity parameters, including periods of physical activity, heart rate, the number of steps taken, the period of sleep time, changes in blood glucose, the amount of carbohydrates and calories taken with food;
training a machine learning algorithm to estimate the user's daily activity basing on the measured parameters of the user's daily activity and a predefined user profile containing the user's gender, age, height and weight;
generating recommendations for maintaining of the user's healthy lifestyle basing on estimation of the user's daily activity received as a result of using the machine learning algorithm; and
displaying the generated recommendations to the user.

12. A system for providing recommendations for maintaining a healthy lifestyle basing on user's daily activity parameters automatically tracked in real time, comprising:
inertial measuring sensors, including an accelerometer and a gyroscope;
a photoplethysmogram sensor;
a blood glucose sensor,
wherein the inertial measuring sensors, the photoplethysmogram sensor and the blood glucose sensor are configured to automatically measure the user's daily activity parameters, including periods of physical activity, changes in blood glucose level, and data of a food intake;
a processing unit configured to train a machine learning algorithm to estimate the user's daily activity basing on the measured parameters of the user's daily activity and a predefined user profile containing the user's gender, age, height and weight;
a storage module configured to store the predefined user profile, the measured parameters of the user's daily activity and estimation of the user's daily activity received as a result of using the machine learning algorithm,
wherein the processing unit is further configured to generate recommendations for maintaining of the user's healthy lifestyle basing on estimation of the user's daily activity, and the storage module is configured to store the generated recommendations,
wherein the system for providing recommendations for maintaining a healthy lifestyle basing on the user's daily activity parameters further comprises a display configured to display the generated recommendations to the user.

13. The system for providing recommendations for maintaining a healthy lifestyle basing on user's daily activity parameters according to claim 12, further comprising a GPS-receiver, configured to determine a user's current geolocation and an additional processing unit, configured to correct the results of estimation of the user's daily activity by said machine learning algorithm basing on the geolocation data of the user.

14. A method for providing recommendations for maintaining a healthy lifestyle basing on user's daily activity parameters automatically tracked in real time, comprising the steps of:
measuring automatically the user's daily activity parameters, including periods of physical activity, changes in blood glucose level, and data of a food intake;
determining indirectly the change in blood glucose level basing on the measured parameters of the user's daily activity, data on ambient sounds, geolocation, user schedules and user profiles containing the user's gender, age, height and weight;
training a machine learning algorithm to estimate the user's daily activity basing on the measured parameters of the user's daily activity, the determined change in blood glucose level and the predefined user profile;
generating recommendations for maintaining of user's healthy lifestyle basing on estimation of the user's daily activity received as a result of using the machine learning algorithm; and
displaying the generated recommendations to the user.

15. A system for providing recommendations for maintaining a healthy lifestyle basing on user's daily activity parameters automatically tracked in real time, comprising:
inertial measuring sensors, including an accelerometer and a gyroscope;
a photoplethysmogram sensor;
wherein the inertial measuring sensors and the photoplethysmogram sensor are configured to measure automatically the user's daily activity parameters, including periods of physical activity, heart rate, the number of steps taken, a sleep time period, the amount of carbohydrates and calories taken with food;
a microphone configured to record ambient sounds;
a GPS-receiver configured to determine a user's current geolocation;
an indirect glucose measurement unit configured to determine indirectly the changes in blood glucose level basing on the measured parameters of the user's daily activity, the data on ambient sounds, the geolocation, a predefined user schedule and a predefined user profile containing the user's gender, age, height and the weight;
a processing unit configured to train a machine learning algorithm to estimate the user's daily activity basing on the measured parameters of the user's daily activity, the determined change in blood glucose level and the predefined user profile;
a storage module configured to store the predefined user schedule, the predefined user profile, the measured parameters of the user's daily activity, the determined change in blood glucose level and estimation of the user's daily activity received as a result of using the machine learning algorithm,
wherein the processing unit is further configured to generate recommendations for maintaining of the user's healthy lifestyle basing on estimation of the user's daily activity, and the storage module is configured to store the generated recommendations,
wherein the system for providing recommendations for maintaining a healthy lifestyle basing on the user's daily activity parameters further comprises a display configured to display the generated recommendations to the user.
